(19) United States
(12) Patent Application Publication
(10) Pub. No.: US 2024/0043799 A1
(43) Pub. Date: Feb. 8, 2024

(54) COMPOSITIONS AND METHODS FOR MODELING HUMAN MICROGLIA

(71) Applicant: The Trustees of Columbia University in the City of New York, New York, NY (US)

(72) Inventors: Wassim Elyaman, Hastings on Hudson, NY (US); Elizabeth M. Bradshaw, Hastings on Hudson, NY (US)

(21) Appl. No.: 18/234,995

(22) Filed: Aug. 17, 2023

Related U.S. Application Data
(63) Continuation of application No. PCT/US22/17023, filed on Feb. 18, 2022.
(60) Provisional application No. 63/151,391, filed on Feb. 19, 2021.

(51) Int. Cl. C12N 5/079; G01N 33/50

(52) U.S. Cl. CPC C12N 5/0622; G01N 33/5058; G01N 2800/52; C12N 2506/115; C12N 2502/081

(57) ABSTRACT

Provided herein are methods and compositions related to the use of human monocyte-derived microglia-like (MDMi) cells. In some embodiments, the methods and compositions provided herein relate to the use of MDMi cells to assess the effect of a clinical intervention on a human subject (e.g., a subject with a neurodegenerative disorder). In some embodiments, the methods and compositions provided herein relate to the use of MDMi cells to stratify human subjects into subgroup populations (e.g., populations that are likely to respond to a clinical intervention or are unlikely to respond to a clinical intervention). In some embodiments, the methods and compositions provided herein relate to the use of MDMi cells to identify candidate neurodegenerative disease biomarkers. In certain embodiments, the methods and compositions provided herein relate to the use of MDMi cells to screen potential therapeutic agents to identify candidate agents for the treatment of a neurodegenerative disease.

### CROSS-REFERENCE TO RELATED APPLICATIONS

This application is a Continuation of International Patent Application PCT/US2022/017023 filed 18 Feb. 2022; which claims priority to U.S. Provisional Patent Application 63/151,391 filed 19 Feb. 2021; each of which is hereby incorporated by reference in its entirety for all purposes.

### BACKGROUND

Neurodegenerative disease is an umbrella term for a range of conditions which primarily affect the neurons in the human brain, which includes Parkinson's disease (PD), Alzheimer's disease (AD), amyotrophic lateral sclerosis (ALS) and Huntington's disease (HD). Microglia are emerging as a key cell type in neurodegenerative diseases and genome-wide associate studies (GWAS) identified several genes that are associated genetically as risk factors with neurodegenerative diseases and these genes are enriched in microglia and other immune cells. In addition to the ongoing inflammatory process in the brain of patients affected with neurodegeneration, recent findings pointed to a dysregulation of peripheral circulating immune cells in neurodegenerative diseases and may contribute to the disease progression. Currently, no neurodegenerative disease is curable, and the treatments available only manage the symptoms or halt the progression of the disease.

A growing body of evidence suggests that inflammation plays a critical role in neurodegenerative diseases of the central nervous system (CNS) including AD, ALS, PD, and HD. Differential immune responses involving the adaptive versus the innate immune system are observed at various stages of neurodegenerative diseases, and may not only drive disease processes but could serve as therapeutic targets.

Microglia are brain-resident macrophages, with important homeostatic functions that provide a supportive environment to neurons. This includes pruning incompetent synapses during development, and clearance of dead cells, misfolded proteins, and other cellular debris (Ransohoff, Nature 532:185-186, 2016). Emerging data suggest that much of the genetic risk of AD and PD plays out in microglia, involving genes including TREM2, CD33, LRRK2, and C9orf72.

Study of microglia has been largely restricted to non-human models (mostly mouse), since availability of fresh primary human microglia is very limited and they cannot be propagated. Moreover, microglia rapidly lose their unique identity when removed from the brain environment and cultured in monoculture in vitro (Butovsky et al., Nat. Neurosci. 17:131-143, 2014). Transformed microglial-like cell lines are by definition highly proliferative and therefore not a good model for understanding a predominantly non-proliferating, differentiated cell type. There is, therefore, a need for an efficient in vitro model of human microglia that can be leveraged for microglia-targeted drug screen and to study the interaction between microglial and neuronal health and their regulation by peripheral inflammatory milieu.

### SUMMARY

Provided herein are methods and compositions related to the use of human monocyte-derived microglia-like (MDMi) cells. In some embodiments, the methods and compositions provided herein relate to the use of MDMi cells to assess the effect of a clinical intervention on a human subject (e.g., a subject with a neurodegenerative disorder). In some embodiments, the methods and compositions provided herein relate to the use of MDMi cells to stratify human subjects into subgroup populations (e.g., populations that are likely to respond to a clinical intervention or are unlikely to respond to a clinical intervention). In some embodiments, the methods and compositions provided herein relate to the use of MDMi cells to identify candidate neurodegenerative disease biomarkers. In certain embodiments, the methods and compositions provided herein relate to the use of MDMi cells to screen potential therapeutic agents to identify candidate agents for the treatment of a neurodegenerative disease.

In certain aspects, provided herein are methods of assessing the effect of a clinical intervention on a human subject.

In some embodiments, such methods comprise the steps of a) obtaining a population of human monocyte-derived microglia-like (MDMi) cells from a human subject undergoing a clinical intervention; b) profiling the population of MDMi cells to obtain an indicator of the effect of the clinical intervention; and c) using the indicator to assess the effect of the clinical intervention.

In some embodiments, such methods comprise the steps of: a) obtaining a first population monocyte-derived microglia-like (MDMi) cells from a human subject undergoing or about to undergo a clinical intervention; b) profiling the first population of MDMi cells to obtain a first indicator; c) subjecting the subject to the clinical intervention; d) obtaining a second population MDMi cells from the human subject undergoing the clinical intervention; e) profiling the second population of MDMi cells to obtain a second indicator; and f) comparing the first indicator with the second indicator to assess the effect of the clinical intervention. In some embodiments, step a) is performed prior to the start of the clinical intervention.

In some embodiments, such methods comprise the steps of a) culturing a population of human monocyte-derived microglia-like (MDMi) cells with a biomaterial from the human subject undergoing a clinical intervention; b) profiling the population of MDMi cells to obtain an indicator of the effect of the clinical intervention; and c) using the indicator to assess the effect of the clinical intervention. Examples of biomaterial from the human subject include, but are not limited to, serum, plasma, immune cells, brain slices, or cerebrospinal fluid. In some embodiments, the first biomaterial is obtained from the subject at an earlier point in the clinical intervention than the second biomaterial (e.g., the first biomaterial is obtained from the subject prior to the start of the clinical intervention).

In certain embodiments, such methods comprise the steps of: a) culturing a first population monocyte-derived microglia-like (MDMi) cells with a first biomaterial from the human subject undergoing or about to undergo a clinical intervention; b) profiling the first population of MDMi cells to obtain a first indicator; c) subjecting the subject to the clinical intervention; d) culturing a second population MDMi cells with a second biomaterial from the human subject undergoing the clinical intervention; e) profiling the second population of MDMi cells to obtain a second indicator; and f) comparing the first indicator with the second indicator to assess the effect of the clinical intervention. Examples of biomaterial from the human subject include, but are not limited to, serum, plasma, immune cells, brain slices, or cerebrospinal fluid. In some embodiments, the first biomaterial is obtained from the subject at an earlier point in the clinical intervention than the second biomaterial (e.g., the first biomaterial is obtained from the subject prior to the start of the clinical intervention).

In some embodiments, the method further comprises the step of co-culturing a population of MDMi cells from the human subject with a population of different cells (e.g., human or mouse neuron cells, astrocyte cells, T cells, oligodendrocyte cells, endothelial cells, or tumor cells). In some embodiments, step the population of different cells are profiled (e.g., to obtain the indicator).

In some embodiments, the method further comprises deriving the MDMi cells from monocytes obtained from the subject. In some embodiments, the population MDMi cells are further differentiated into cells resembling microglial subtypes.

In some embodiments, the indicator is compared to a reference indicator (e.g., the reference indicator is an indicator obtained from the subject at an earlier time (e.g., the reference indicator was obtained from the subject prior to the start of the clinical intervention and/or at an earlier time point during the intervention.)).

In some embodiments, if the clinical intervention is determined to be effective then the clinical intervention continues to be administered to the subject. In some embodiments, if the clinical intervention is determined to be ineffective then the clinical intervention is no longer administered to the subject. In some embodiments, if the clinical intervention is determined to be ineffective then a different clinical intervention is administered to the subject.

In certain aspects, provided herein are methods of determining a subgroup population to which a human subject belongs (e.g., a subgroup population likely to respond to a clinical intervention, a subgroup population unlikely to respond to a clinical intervention).

In certain embodiments, such methods comprise the steps of: a) obtaining a population of human monocyte-derived microglia-like (MDMi) cells from a human subject; b) profiling the population of MDMi cells to obtain an indicator of the subgroup population of the subject; and c) using the indicator to determine the subgroup population to which the human subject belongs.

In some embodiments, such methods comprise the steps of: a) culturing a population of human monocyte-derived microglia-like (MDMi) cells with a biomaterial from the human subject; and b) profiling the population of MDMi cells to obtain an indicator of the subgroup population of the subject; and c) using the indicator to determine the subgroup population to which the human subject belongs. Examples of biomaterial from the human subject include but are not limited to serum, plasma, immune cells, brain slices, or cerebrospinal fluid.

In some embodiments, the subgroup population may be a population of likely responders to a clinical intervention or a population of likely non-responders to a clinical intervention.

In some embodiments, the method further comprises the step of co-culturing the population of MDMi cells from the human subject with a population of different cells (e.g., neuron cells, astrocyte cells, T cells, oligodendrocyte cells, endothelial cells, or tumor cells). In some embodiments, step b) further comprises profiling the population of different cells to obtain the indicator.

In some embodiments, step a) comprises deriving the MDMi cells from monocytes obtained from the subject. In some embodiments, the population MDMi cells are further differentiated into cells resembling microglial subtypes prior to step b).

In some embodiments, the subject is administered the clinical intervention if the subject is determined to belong to the subgroup population of likely responders to the clinical intervention. In some embodiments, the subject is not administered the clinical intervention if the subject is determined to belong to the subgroup population of likely non-responders to the clinical intervention. In some embodiments, the subject is administered the clinical intervention if the subject is determined to belong to the subgroup population of likely responders to the clinical intervention. In some embodiments, the subject is not administered the clinical intervention if the subject is determined to belong to the subgroup population of likely non-responders to the clinical intervention.

In some embodiments, step c) comprises comparing the indicator to a reference indicator.

In certain aspects, provided herein are methods of determining whether a human subject is likely to respond to a pharmaceutical agent.

In some embodiments, such methods comprise the steps of: a) obtaining a population of human monocyte-derived microglia-like (MDMi) cells from a human subject; b) contacting the MDMi cells with a pharmaceutical agent to obtain an indicator of whether the subject is likely to respond to the pharmaceutical agent; and c) using the indicator to determine whether the subject is likely to respond to the pharmaceutical agent. Pharmaceutical agents include but are not limited to a small molecule agent, a polypeptide agent, a protein agent, an antibody or antigen-binding fragment thereof agent, or a nucleic acid agent.

In certain embodiments, such methods comprise the steps of: a) culturing a population of human monocyte-derived microglia-like (MDMi) cells with a biomaterial from the human subject; b) contacting the MDMi cells with a pharmaceutical agent to obtain an indicator of whether the subject is likely to respond to the pharmaceutical agent; and c) using the indicator to determine whether the subject is likely to respond to the pharmaceutical agent. Examples of biomaterial from the human subject include but are not limited to serum, plasma, immune cells, brain slices, or cerebrospinal fluid.

In some embodiments, the method further comprises the step of co-culturing the population of MDMi cells from the human subject with a population of different cells (e.g., neuron cells, astrocyte cells, T cells, oligodendrocyte cells, endothelial cells, or tumor cells). In some embodiments, step b) further comprises profiling the population of different cells to obtain the indicator.

In some embodiments, step a) comprises deriving the MDMi cells from monocytes obtained from the subject. In some embodiments, the population MDMi cells are further differentiated into cells resembling microglial subtypes prior to step b).

In some embodiments, if the subject is determined to be likely to respond to the pharmaceutical agent the pharmaceutical agent is administered to the subject. In some embodiments, if the subject is not determined to be likely to respond to the pharmaceutical agent the pharmaceutical agent is not administered to the subject

In some embodiments, step c) comprises comparing the indicator to a reference indicator.

In some embodiments of the methods provided herein, the subject has a neurodegenerative disorder (e.g., Alzheimer's disease (AD), amyotrophic lateral sclerosis (ALS), Parkinson's disease (PD), Huntington's disease (HD), epilepsy, frontotemporal dementia (FTD), Friedreich ataxia, Lewy body disease, spinal muscular atrophy, multiple sclerosis, progressive multifocal leukoencephalopathy, or leukodystrophy).

Examples of clinical intervention include but are not limited to cell-based therapy, small molecule therapy, biologic therapy, or surgery. In some embodiments, the clinical intervention comprises the administration of a pharmaceutical agent (e.g., administration of a small molecule agent, a polypeptide agent, a protein agent, an antibody or antigen-binding fragment thereof agent, a nucleic acid agent) to the subject.

In some embodiments, the indicator comprises measures of protein expression, transcription, epigenetic modifications, uptake ability, chemokine production, cytokine production, cell morphological analysis, cell trafficking, or cell viability.

In certain aspects, provided herein are methods of assessing the efficacy of a pharmaceutical agent. In certain embodiments, such methods comprise the steps of: a) contacting a population of human monocyte-derived microglia-like (MDMi) cells with a pharmaceutical agent to obtain an indicator of the efficacy of the pharmaceutical agent; and b) using the indicator to determine the efficacy of the pharmaceutical agent. Pharmaceutical agents may include a small molecule agent, a polypeptide agent, a protein agent, an antibody or antigen-binding fragment thereof agent, or a nucleic acid agent.

In some embodiments, the method further comprises the step of co-culturing the population of MDMi cells from the human subject with a population of different cells (e.g., neuron cells, astrocyte cells, T cells, oligodendrocyte cells, endothelial cells, or tumor cells). In some embodiments, step a) further comprises profiling the population of different cells to obtain the indicator.

In some embodiments, the efficacy of the pharmaceutical agent is its efficacy in the treatment of a neurodegenerative disorder (e.g., Alzheimer's disease (AD), amyotrophic lateral sclerosis (ALS), Parkinson's disease (PD), Huntington's disease (HD), epilepsy, frontotemporal dementia (FTD), Friedreich ataxia, Lewy body disease, Spinal muscular atrophy, Multiple Sclerosis, Progressive Multifocal Leukoencephalopathy, or Leukodystrophy).

In some embodiments, the population MDMi cells are further differentiated into cells resembling microglial subtypes prior to step a).

In some embodiments, the indicator comprises measures of protein expression, transcription, uptake ability, cytokine production, chemokine production, epigenetic modifications, cell morphological analysis, cell trafficking, or cell viability. In some embodiments, step b) comprises comparing the indicator to a reference indicator.

In certain aspects, provided herein are methods of screening a library of pharmaceutical agents. In certain embodiments, such methods comprise the steps of: a) contacting populations of human monocyte-derived microglia-like (MDMi) cells with pharmaceutical agents from the library of pharmaceutical agents to obtain an indicators of the efficacy of the pharmaceutical agents; and b) using the indicators to select pharmaceutical agents from the library of pharmaceutical agents. Pharmaceutical agents may include a small molecule agent, a polypeptide agent, a protein agent, an antibody or antigen-binding fragment thereof agent, or a nucleic acid agent.

In some embodiments, the method further comprises the step of co-culturing the population of MDMi cells from the human subject with a population of different cells (e.g., neuron cells, astrocyte cells, T cells, oligodendrocyte cells, endothelial cells, or tumor cells). In some embodiments, step a) further comprises profiling the populations of different cells to obtain the indicators.

In some embodiments, the efficacy of the pharmaceutical agents are their efficacy in the treatment of a neurodegenerative disorder (e.g., Alzheimer's disease (AD), amyotrophic lateral sclerosis (ALS), Parkinson's disease (PD), Huntington's disease (HD), epilepsy, frontotemporal dementia (FTD), Friedreich ataxia, Lewy body disease, Spinal muscular atrophy, Multiple Sclerosis, Progressive Multifocal Leukoencephalopathy, or Leukodystrophy).

In some embodiments, the populations MDMi cells are further differentiated into cells resembling microglial subtypes prior to step a).

In some embodiments, the indicator comprises measures of protein expression, transcription, uptake ability, cytokine production, chemokine production, epigenetic modifications, cell morphological analysis, cell trafficking, or cell viability. In some embodiments, step b) comprises comparing the indicator to a reference indicator.

In certain aspects, provided herein are methods of identifying candidate neurodegenerative disease biomarkers. In certain embodiments, such methods comprise the steps of: a) modifying and/or silencing a test gene in a population of human monocyte-derived microglia-like (MDMi) cells; b) obtaining an indicator of neuronal health in the MDMi cells; and c) using the indicator to identify the test gene as a candidate neurodegenerative disease biomarker.

In some embodiments, the test gene is silenced by contacting the population of MDMi cells using an inhibitory RNA molecule. In some embodiments, the test gene is modified or silenced by inducing a mutation in the genome of the cells of the population of MDMi cells. In some embodiments, the mutation in the genome of the cells is induced by contacting the population of MDMi cells with a CRISPR/Cas agent.

In some embodiments, the indicator of neuronal health is determined by measuring a level of synaptic formation, synaptic pruning, synaptic density, microglia uptake function, and/or neuronal viability. In some embodiments, the level of synaptic pruning is determined by measuring a level of a amyloid peptide and/or alpha-synuclein. In some embodiments, the level of synaptic pruning is determined by measuring a level of phosphorylation of tau protein. In some embodiments, the level of neuronal viability is determined by measuring a level of cleaved caspase-3.

In some embodiments, the method further comprises the step of co-culturing the population of MDMi cells from the human subject with a population of different cells (e.g., neuron cells, astrocyte cells, T cells, oligodendrocyte cells, endothelial cells, or tumor cells). In some embodiments, step b) further comprises profiling the population of different cells to obtain the indicator.

In some embodiments, the MDMi cells are derived from a subject with a neurodegenerative disorder (e.g., Alzheimer's disease (AD), amyotrophic lateral sclerosis (ALS), Parkinson's disease (PD), Huntington's disease (HD), epilepsy, frontotemporal dementia (FTD), Friedreich ataxia, Lewy body disease, Spinal muscular atrophy, Multiple Sclerosis, Progressive Multifocal Leukoencephalopathy, or Leukodystrophy). In some embodiments, the population MDMi cells are further differentiated into cells resembling microglial subtypes prior to step b).

In some embodiments, the indicator comprises measures of protein expression, transcription, uptake ability, cytokine production, epigenetic modifications, chemokine production, cell morphological analysis, cell trafficking, or cell viability. In some embodiments, step c) comprises comparing the indicator to a reference indicator.

In certain aspects, provided herein are methods of identifying a therapeutic agent for treatment of a neurodegenerative disease. In some embodiments, such methods comprise the steps of: a) co-culturing a human monocyte-derived microglia-like cell with rodent or human-derived primary neuronal cells in the presence of a fluid sample from a patient with a neurodegenerative disease; b) contacting test compounds with the product of step a); and c) identifying a test compound that protects or increases synaptic formation, regulates synaptic pruning or microglia uptake function or neuronal viability in comparison to a control compound. In certain embodiments the neurodegenerative disease is Alzheimer's disease, Huntington's disease, amyotrophic lateral sclerosis or Parkinson's disease. In some embodiments the sample comprises plasma, serum or cerebrospinal fluid from said patient.

In some embodiments, the present disclosure also provides a composition for treatment of a neurodegenerative disease identified according to a method comprising: a) co-culturing a human monocyte-derived microglia-like cell with rodent or human-derived primary neuronal cells in the presence of a fluid sample from a patient with a neurodegenerative disease; b) contacting test compounds with the product of step a); and c) identifying a test compound that protects or increases synaptic formation, regulates synaptic pruning or microglia uptake function or neuronal viability in comparison to a control compound. In certain embodiments the neurodegenerative disease is Alzheimer's disease, Huntington's disease, amyotrophic lateral sclerosis or Parkinson's disease.

In certain embodiments, the present disclosure additionally provides a method of treating a neurodegenerative disease in a patient, comprising administering to said patient a therapeutically effective amount of a composition identified according to a method comprising: a) co-culturing a human monocyte-derived microglia-like cell with rodent or human-derived primary neuronal cells in the presence of a fluid sample from a patient with a neurodegenerative disease; b) contacting test compounds with the product of step a); and c) identifying a test compound that protects or increases synaptic formation, regulates synaptic pruning or microglia uptake function or neuronal viability in comparison to a control compound. In certain embodiments the neurodegenerative disease is Alzheimer's disease, Huntington's disease, amyotrophic lateral sclerosis or Parkinson's disease.

In certain embodiments, the present disclosure further provides a system for analyzing the effect of a test compound on a neurodegenerative disease, comprising a human monocyte-derived microglia-like cell, a murine primary neuronal cell, a human iPSC-derived neuron, a sample from a patient with a neurodegenerative disease, and a test compound. In certain embodiments the system further comprises a control compound. In various embodiments the neurodegenerative disease is Alzheimer's disease, amyotrophic lateral sclerosis, Huntington's disease or Parkinson's disease. In some embodiments the sample comprises plasma, serum or cerebrospinal fluid from said patient.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 shows polarization of monocytes to monocyte-derived microglia-like cells (MDMi) that resemble microglia morphologically and express microglial gene signature. Peripheral human monocytes from young healthy subjects were incubated with polarizing cytokines and differentiated to MDMi. Microscopy analysis shows that MDMi express microglia specific genes, P2RY12 (top panels), TMEM119 (middle panels) and PU.1 (bottom panels) as analyzed by immunofluorescence techniques.

FIG. 2 shows a schematic representation of the small molecule screen of small compounds using a xenogeneic co-culture model. Human monocyte-derived microglia-like cells (MDMi) are generated as described recently (Ryan et al., Sci. Transl. Med. 9:(doi: 10.1126/scitranslmed.aai7635), 2017) using PBMCs from healthy subjects and are co-cultured with rat primary cortical neurons (NeuN is a marker of neurons, GFAP marks astrocytes and DAPI stains nuclei) in a 96-well plate. On day 7, cells are exposed to fibrilized FITC-labeled A-beta 1-42 (2.5 μM) in the presence or absence of small molecules (n=200, 1 μM) for 24 hours followed by assessment of 1) neuronal survival (activated caspase-3) , 2) tau phosphorylation (p-tau, AT8 site: serine 202 and threonine 205), and 3) MDMi uptake and using the Celigo high throughput micro-well image cytometer (Nexcelom™).

FIG. 3 shows the addition of a small molecule targeting CD45 leads to a reduction in phosphorylated tau (serine 202) detected using AT8 antibody in neurons in the presence of Amyloid-beta stress.

FIG. 4 are exemplary dot plots showing luminex analysis of a panel of 10 cytokines and chemokines of healthy MDMi exposed to serum from medically-controlled and medically-refractory patients. Media were collected after 24 h of treatment and were measured using Luminex kit. Serum from healthy control (HC) were used. Each dot represents an MDMi culture. Statistical analysis was performed using Student t test.

FIG. 5 are exemplary micrographs showing uptake function of healthy MDMi exposed to serum from medically-controlled and medically-refractory patients by analysis of FITC-labelled Dextran (green). MDMi cells are labelled using anti-CD40 antibody (red). Dead cells are labelled with AO/PI (purple) and nuclei are stained with DAPI (blue). Each dot represents an MDMi culture. A group of MDMi cultures was not exposed to serum and used as a negative control.

FIG. 6 are exemplary micrographs and dot plot showing uptake function of healthy MDMi exposed to CSF from Alzheimer's patients. Representative imaged of MDMi uptake is shown (left panel). Cell uptake function was calculated, and data are presented as mean fluorescence intensity (right panel) of amyloid beta signal (green) in MDMi cells (red). Each dot represents an MDMi culture.

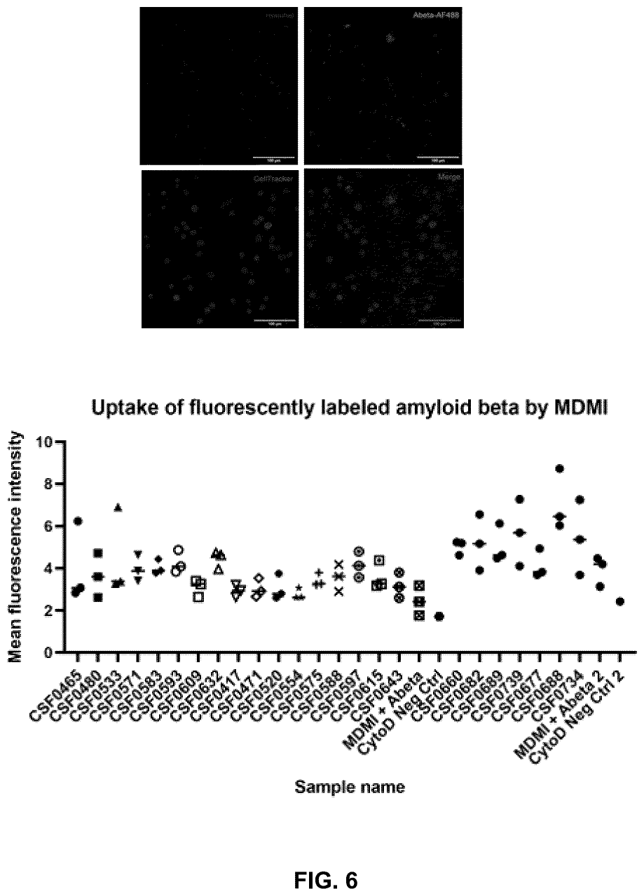

### DETAILED DESCRIPTION

The present disclosure is based, in part, on the development of an in vitro model system of human monocyte-derived microglia-like cells (MDMi). In some embodiments, such cells can be co-cultured with either human (induced pluripotent stem cells (iPSC)-derived-neurons) or murine primary neuronal cells in the presence of plasma, serum or CSF (cerebrospinal fluid) from patients with a neurodegenerative disease such as Alzheimer's disease (AD), amyotrophic lateral sclerosis (ALS), Parkinson's disease (PD), or Huntington's disease (HD), or control samples from healthy donors. In some embodiments, this model can be used to study the effects of microglia-specific genes on synaptic formation, neuronal viability and function (e.g., using small molecules, biologics and/or by gene deletion strategy). To study the influence of gene risks on the response to drug screen, MDMi can be generated from a cohort of healthy genome-wide genotyped subjects. Cell cultures can be analyzed, for example, using a 4-color imaging system (Celigo™) that is able to scan a 96-well plate in seven minutes.

The model described herein can be used for drug screening applications for neurodegenerative disease therapeutic candidates. The model described herein can be used in preclinical research to identify drug targets and molecular pathways for neurodegenerative diseases. The model described herein can also be used to assess the effect of a clinical intervention on a human subject (e.g., a subject with a neurodegenerative disorder). The model described herein can also be used to stratify human subjects into subgroup populations (e.g., populations that are likely to respond to a clinical intervention or are unlikely to respond to a clinical intervention). The model described herein can further be used to identify candidate neurodegenerative disease biomarkers.

Disclosed herein are methods of assessing the effect of clinical intervention on a human subject, a subgroup population to which a human subject belongs, determining whether a human subject is likely to respond to a pharmaceutical agent, assessing the efficacy of a pharmaceutical agent, and screening a library of pharmaceutical agents.

### Monocyte-Derived Microglia-Like Cells (MDMi) Cell Cultures

In certain aspects, the methods described herein comprise the use of monocyte-derived microglia-like cells (MDMi) cell cultures. Generation of MDMi cells is described in Ryan K J, White C C, Patel K, et al. A human microglia-like cellular model for assessing the effects of neurodegenerative disease gene variants. Sci Transl Med. 2017;9(421):eaai7635, incorporated herein by reference.

In certain embodiments, MDMi cells of the methods described herein are derived from monocytes obtained from a subject.

In certain embodiments, the MDMi cells of the methods described herein are co-cultured with a population of different cells. In some embodiments, the MDMi cells are co-cultured with neuron cells, astrocyte cells, T cells, oligodendrocyte cells, endothelial cells, or tumor cells). In some embodiments, the MDMi cells are co-cultured with a central nervous system (CNS) cell (e.g., brain cells and/or spinal cord cells). In some embodiments, the MDMi cells are co-cultured with a stem cell (e.g., a hematopoietic cell). In some embodiments, the MDMi cells are co-cultured with a vascular cell. In some embodiments, the MDMi cells are co-cultured with an ocular cell. In some embodiments, the MDMi cells are co-cultured with an immune cell (e.g., antigen-presenting cells, including professional antigen presenting cells). Such immune cells can include macrophages (e.g., bone marrow derived macrophages), dendritic cells (e.g., bone marrow derived plasmacytoid dendritic cells and/or bone marrow derived myeloid dendritic cells), or monocytes (e.g., human peripheral blood monocytes).

In certain embodiments, the MDMi cells of the methods described herein are cultured with a biomaterial from a subject. In some embodiments, the biomaterial is serum, plasma, immune cells, brain slices, or cerebrospinal fluid.

In certain embodiments, the method described herein comprise profiling a population of MDMi cells to obtain an indicator. In some embodiments, the indicator comprises measures of protein expression, transcription, epigenetic modifications, uptake ability, chemokine production, cytokine production, cell morphological analysis, cell trafficking, or cell viability.

In some preferred embodiments, the cytokine and/or chemokine is IFNγ, IL-12p70, IL-1α, IL-6, IL-8, MCP1, MIP1α, MIP1β, and/or TNFα.

In some embodiments, the cytokine is an anti-inflammatory cytokine. Anti-inflammatory cytokines may include, but are not limited to, IL-10, IL-13, IL-9, IL-4, IL-5, TGFβ, and combinations thereof. Other exemplary anti-inflammatory cytokines are known in the art.

In some embodiments, the cytokine is a pro-inflammatory cytokine. Pro-inflammatory cytokines may include, but are not limited to, IFNγ, IL-12p70, IL-1α, IL-6, IL-8, MCP1, MIP1α, MIP1β, TNFα, and combinations thereof. Other exemplary pro-inflammatory cytokines are known in the art.

In certain embodiments, cytokine secretion can be determined by any appropriate method known in the art. In some embodiments, cytokine secretion is determined by ELISA. In some embodiments, cytokine secretion is determined by MSD assay. In some embodiments, cytokine secretion is determined by Luminex Assay.

In some embodiments, the ability of the cell type to secrete the cytokine of interest is confirmed, e.g., by use of positive controls, e.g., contacting an agent that is known to induce the cytokine with cells of the cell type, and/or assaying the cells for cytokine mRNA or protein expression.

In certain embodiments, the indicator is an indicator of neuronal health. In some embodiments, the indicator of neuronal health is determined by measuring a level of synaptic formation, synaptic pruning, synaptic density, microglia uptake function, and/or neuronal viability. In some embodiments, the level of synaptic pruning is determined by measuring a level of a amyloid peptide and/or alpha-synuclein. In some embodiments, the level of synaptic pruning is determined by measuring a level of phosphorylation of tau protein. In some embodiments, the level of neuronal viability is determined by measuring a level of cleaved caspase-3.

### Neurodegenerative Disorders

In certain embodiments, the methods described herein relate to neurodegenerative disorders (e.g., screening agents for the ability to treat a neurodegenerative disease, assessing human subjects who have a neurodegenerative disease).

In some embodiments, the neurodegenerative is Alzheimer's disease (AD), amyotrophic lateral sclerosis (ALS), Parkinson's disease (PD), Huntington's disease (HD), epilepsy, frontotemporal dementia (FTD), Friedreich ataxia, Lewy body disease, Spinal muscular atrophy, Multiple Sclerosis, Progressive Multifocal Leukoencephalopathy, or Leukodystrophy), dementia, prion disease, macular degeneration, motor neuron diseases (MND), spinocerebellar ataxia, spinal muscular atrophy, dystonia, idiopathicintracranial hypertension, epilepsy, nervous system disease, central nervous system disease, movement disorders, encephalopathy, peripheral neuropathy, post-operative cognitive dysfunction, AIDS dementia complex, adrenoleukodystrophy, Alexander disease, Alper's disease, ataxia telangiectasia, Batten disease, bovine spongiform encephalopathy (BSE), Canavan disease, corti co basal degeneration, Creutzfeldt-Jakob disease, dementia with Lewy bodies, fatal familial insomnia, frontotemporal lobar degeneration, Kennedy's disease, Krabbe disease, Lyme disease, Machado-Joseph disease, multiple system atrophy, neuroacanthocytosis, Niemann-Pick disease, Pick's disease, primary lateral sclerosis, progressive supranuclear palsy, Refsum disease, Sandhoff disease, diffuse myelinoclastic sclerosis, spinocerebellar ataxia, subacute combined degeneration of spinal cord, tabes dorsalis, Tay-Sachs disease, toxic encephalopathy, transmissible spongiform encephalopathy, and wobbly hedgehog syndrome.

### Methods of Assessing Clinical Intervention

In certain aspects, provided herein are methods of assessing the effect of a clinical intervention on a human subject. In certain embodiments, the clinical intervention can be any clinical intervention for a neurodegenerative disease. Examples of clinical interventions include but are not limited to, cell-based therapy, small molecule therapy, biologic therapy, focus ultrasound, gene therapy, or surgery. In some embodiments, the method comprises the steps of a) obtaining a population of human monocyte-derived microglia-like (MDMi) cells from a human subject undergoing a clinical intervention; b) profiling the population of MDMi cells to obtain an indicator of the effect of the clinical intervention; and c) using the indicator to assess the effect of the clinical intervention.

In some embodiments, the method comprises the steps of: a) obtaining a first population monocyte-derived microglia-like (MDMi) cells from a human subject undergoing or about to undergo a clinical intervention; b) profiling the first population of MDMi cells to obtain a first indicator; c) subjecting the subject to the clinical intervention; d) obtaining a second population MDMi cells from the human subject undergoing the clinical intervention; e) profiling the second population of MDMi cells to obtain a second indicator; and f) comparing the first indicator with the second indicator to assess the effect of the clinical intervention.

In some embodiments, the method comprises the steps of the steps of a) culturing a population of human monocyte-derived microglia-like (MDMi) cells with a biomaterial (e.g., serum, plasma, immune cells, brain slices, or cerebrospinal fluid) from the human subject undergoing a clinical intervention; b) profiling the population of MDMi cells to obtain an indicator of the effect of the clinical intervention; and c) using the indicator to assess the effect of the clinical intervention.

In some embodiments, the method comprises the steps of: a) culturing a first population monocyte-derived microglia-like (MDMi) cells with a first biomaterial (e.g., serum, plasma, immune cells, brain slices, or cerebrospinal fluid) from the human subject undergoing or about to undergo a clinical intervention; b) profiling the first population of MDMi cells to obtain a first indicator; c) subjecting the subject to the clinical intervention; d) culturing a second population MDMi cells with a second biomaterial from the human subject undergoing the clinical intervention; e) profiling the second population of MDMi cells to obtain a second indicator; and f) comparing the first indicator with the second indicator to assess the effect of the clinical intervention.

In some embodiments, if the clinical intervention is determined to be effective then the clinical intervention continues to be administered to the subject. In some embodiments, if the clinical intervention is determined to be ineffective then the clinical intervention is no longer administered to the subject. In some embodiments, if the clinical intervention is determined to be ineffective then a different clinical intervention is administered to the subject.

### Methods of Determining Population Subgroups

In certain aspects, provided herein are methods of determining a subgroup population to which a human subject belongs. In some embodiments, the subgroup population may be a population of likely responders to a clinical intervention disclosed herein and/or a population of likely non-responders to a clinical intervention.

In some embodiments, the method comprises the steps of: a) obtaining a population of human monocyte-derived microglia-like (MDMi) cells from a human subject; b) profiling the population of MDMi cells to obtain an indicator of the subgroup population of the subject; and c) using the indicator to determine the subgroup population to which the human subject belongs.

In some embodiments, the method comprises the steps of: a) culturing a population of human monocyte-derived microglia-like (MDMi) cells with a biomaterial from the human subject; and b) profiling the population of MDMi cells to obtain an indicator of the subgroup population of the subject; and c) using the indicator to determine the subgroup population to which the human subject belongs. Examples of biomaterial from the human subject include but are not limited to serum, plasma, immune cells, brain slices, or cerebrospinal fluid.

### Methods of Determining Likely Responses to Pharmaceutical Agents

In certain aspects, provided herein are methods of determining whether a human subject is likely to respond to a pharmaceutical agent. In some embodiments, the pharmaceutical agent is a pharmaceutical agent for the treatment of a neurodegenerative disease. Pharmaceutical agents may include a small molecule agent, a polypeptide agent, a protein agent, an antibody or antigen-binding fragment thereof agent, gene therapy or a nucleic acid agent.

In some embodiments, the method comprises the steps of: a) obtaining a population of human monocyte-derived microglia-like (MDMi) cells from a human subject; b) contacting the MDMi cells with a pharmaceutical agent to obtain an indicator of whether the subject is likely to respond to the pharmaceutical agent; and c) using the indicator to determine whether the subject is likely to respond to the pharmaceutical agent.

In some embodiments, the method comprises the steps of: a) culturing a population of human monocyte-derived microglia-like (MDMi) cells with a biomaterial from the human subject; b) contacting the MDMi cells with a pharmaceutical agent to obtain an indicator of whether the subject is likely to respond to the pharmaceutical agent; and c) using the indicator to determine whether the subject is likely to respond to the pharmaceutical agent. Examples of biomaterial from the human subject include but are not limited to serum, plasma, immune cells, brain slices, or cerebrospinal fluid.

In some embodiments, if the subject is determined to be likely to respond to the pharmaceutical agent the pharmaceutical agent is administered to the subject. In some embodiments, if the subject is not determined to be likely to respond to the pharmaceutical agent the pharmaceutical agent is not administered to the subject.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into a RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see, e.g., Sambrook and Russel, Condensed Protocols from Molecular Cloning: A Laboratory Manual, Cold Spring Harbor Laboratory Press, 2006, ISBN-10: 0879697717; Ausubel et al., Short Protocols in Molecular Biology, 5th ed., Current Protocols, 2002, ISBN-10: 0471250929; Sambrook and Russel, Molecular Cloning: A Laboratory Manual, 3d ed., Cold Spring Harbor Laboratory Press, 2001, ISBN-10: 0879695773; Elhai and Wolk, 1988. Meth. Enzymol. 167:747-754, 1988).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A constructs of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal or a plant into which a heterologous nucleic acid molecule has been introduced. The nucleic acid molecule can be stably integrated into the genome as generally known in the art. Integration of the nucleic acid molecule can be confirmed by methods including, but not limited to, Polymerase Chain Reaction (PCR). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above required percent identities and retaining a required activity of the expressed protein is within the skill of the art. For example, directed evolution and rapid isolation of mutants can be according to methods described in references including, but not limited to, Link et al., Nature Rev. 5:680-688, 2007; Sanger et al., Gene 97:119-123, 1991; Ghadessy et al., Proc. Natl. Acad. Sci. USA 98:4552-4557, 2001. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example the exchange of Glu by Asp, Gln by Asn, Val by Ile, Leu by Ile, and Ser by Thr. Deletion is the replacement of an amino acid by a direct bond.

Positions for deletions include the termini of a polypeptide and linkages between individual protein domains. Insertions are introductions of amino acids into the polypeptide chain, a direct bond formally being replaced by one or more amino acids. Amino acid sequence can be modulated with the help of art-known computer simulation programs that can produce a polypeptide with, for example, improved activity or altered regulation. On the basis of this artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41(fraction G/C content)−0.63(% formamide)−(600/l). Furthermore, the Tₘ of a DNA:DNA hybrid is decreased by 1-1.5° C. for every 1% decrease in nucleotide identity (see, e.g., Sambrook and Russel, 2006).

Host cells can be transformed using a variety of standard techniques known to the art (see, e.g., Sambrook and Russel, Condensed Protocols from Molecular Cloning: A Laboratory Manual, Cold Spring Harbor Laboratory Press, 2006, ISBN-10: 0879697717; Ausubel et al., Short Protocols in Molecular Biology, 5th ed., Current Protocols, 2002, ISBN-10: 0471250929; Sambrook and Russel, Molecular Cloning: A Laboratory Manual, 3d ed., Cold Spring Harbor Laboratory Press, 2001, ISBN-10: 0879695773; Elhai and Wolk, Meth. Enzymol. 167:747-754, 1988). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier, Protein Expr. Purif. 41:207-234, 2005; Gellissen, ed., Production of Recombinant Proteins: Novel Microbial and Eukaryotic Expression Systems, Wiley-VCH, 2005, ISBN-10: 3527310363; Baneyx, Protein Expression Technologies, Taylor & Francis, 2004, ISBN-10: 0954523253).

Methods of down-regulation or silencing genes are known in the art. For example, expressed protein activity can be down-regulated or eliminated using antisense oligonucleotides, protein aptamers, nucleotide aptamers, and RNA interference (RNAi) (e.g., small interfering RNAs (siRNA), short hairpin RNA (shRNA), and micro RNAs (miRNA) (see, e.g., Fanning and Symonds, Handb. Exp. Pharmacol. 173:289-303G, 2006, describing hammerhead ribozymes and small hairpin RNA; Helene et al., Ann. N. Y. Acad. Sci. 660:27-36, 1992; Maher Bioassays 14:807-15, 1992, describing targeting deoxyribonucleotide sequences; Lee et al., Curr. Opin. Chem. Biol. 10:1-8, 2006, describing aptamers; Reynolds et al., Nat. Biotechnol. 22:326-330, 2004, describing RNAi; Pushparaj and Melendez, Clin. Exp. Pharmacol. Physiol. 33:504-510, 2006, describing RNAi; Dillon et al., Annu. Rev. Physiol. 67, 147-173, 2005, describing RNAi; Dykxhoorn and Lieberman, Annu. Rev. Med. 56, 401-423, 2005, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

### Formulation

The agents and compositions described herein can be formulated by any conventional manner using one or more pharmaceutically acceptable carriers or excipients as described in, for example, Remington's Pharmaceutical Sciences (A.R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

In some embodiments, the pharmaceutical agent described herein is a small molecule agent, a polypeptide agent, a protein agent, an antibody or antigen-binding fragment thereof agent, or a nucleic acid agent.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc., Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Therapeutic Methods

Also provided is a process of treating a neurodegenerative disease in a subject in need thereof by administration of a therapeutically effective amount of an agent identified via screening assays described herein.

Methods described herein are generally performed on a subject in need thereof. A subject in need of the therapeutic methods described herein can be a subject having, diagnosed with, suspected of having, or at risk for developing a neurodegenerative disease, such as, but not limited to, Alzheimer's disease (AD), Parkinson's disease (PD), amyotrophic lateral sclerosis (ALS) or Huntington's disease (HD). A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and chickens, and humans. For example, the subject can be a human subject.

Generally, a safe and effective amount of an agent identified via screening assays described herein is, for example, that amount that would cause the desired therapeutic effect in a subject while minimizing undesired side effects. In various embodiments, an effective amount of an agent identified via screening assays described herein can substantially inhibit a neurodegenerative disease, ameliorate a neurodegenerative disease, slow the progress of a neurodegenerative disease, or limit the development of a neurodegenerative disease.

According to the methods described herein, administration can be parenteral, pulmonary, oral, topical, intradermal, intramuscular, intraperitoneal, intravenous, subcutaneous, intranasal, epidural, ophthalmic, buccal, or rectal administration.

When used in the treatments described herein, a therapeutically effective amount of an agent identified via screening assays described herein can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to treat a neurodegenerative disease.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see, e.g., Koda-Kimble et al., Applied Therapeutics: The Clinical Use of Drugs, Lippincott Williams & Wilkins, 2004, ISBN 0781748453; Winter, Basic Clinical Pharmacokinetics, 4^{th} ed., Lippincott Williams & Wilkins, 2003, ISBN 0781741475; Sharqel, Applied Biopharmaceutics & Pharmacokinetics, McGraw-Hill/Appleton & Lange, 2004, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of an agent identified via screening assays described herein can occur as a single event or over a time course of treatment. For example, an agent identified via screening assays described herein can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for a neurodegenerative disease.

An agent identified via screening assays described herein can be administered simultaneously or sequentially with another agent, such as an antibiotic, an anti-inflammatory, or another agent. For example, an agent identified via screening assays described herein can be administered simultaneously with another agent, such as an antibiotic or an anti-inflammatory. Simultaneous administration can occur through administration of separate compositions, each containing one or more of an agent identified via screening assays described herein, an antibiotic, an anti-inflammatory, or another agent. Simultaneous administration can occur through administration of one composition containing two or more of an agent identified via screening assays described herein, an antibiotic, an anti-inflammatory, or another agent. An agent identified via screening assays described herein can be administered sequentially with an antibiotic, an anti-inflammatory, or another agent. For example, an agent identified via screening assays described herein can be administered before or after administration of an antibiotic, an anti-inflammatory, or another agent.

### Administration

Agents and compositions described herein can be administered according to methods described herein in a variety of means known to the art. The agents and composition can be used therapeutically either as exogenous agents or materials or as endogenous agents or materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intramuscular, intraperitoneal, intravenous, subcutaneous, intranasal, epidural, ophthalmic, buccal, or rectal administration.

Agents and compositions described herein can be administered in a variety of methods well known in the art. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include, but are not limited to, polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds., Polymers in Drug Delivery, CRC,2006, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to non-target tissues; decrease irritation caused by the agent; decrease toxicity due to high initial doses of the agent; alter the immunogenicity of the agent; decrease dosage frequency, improve taste of the product; or improve shelf life of the product.

### Screening

Also provided are methods for screening potential pharmaceutical agents (e.g., potential pharmaceutical agents for the treatment of a neurodegenerative disorder). Pharmaceutical agents may include a small molecule agent, a polypeptide agent, a protein agent, an antibody or antigen-binding fragment thereof agent, or a nucleic acid agent.

In certain aspects, provided herein are methods of assessing the efficacy of a pharmaceutical agent, the method comprising the steps of: a) contacting a population of human monocyte-derived microglia-like (MDMi) cells with a pharmaceutical agent to obtain an indicator of the efficacy of the pharmaceutical agent; and b) using the indicator to determine the efficacy of the pharmaceutical agent. Pharmaceutical agents may include a small molecule agent, a polypeptide agent, a protein agent, an antibody or antigen-binding fragment thereof agent, or a nucleic acid agent.

In certain aspects, provided herein are methods of screening a library of pharmaceutical agents, the method comprising the steps of: a) contacting populations of human monocyte-derived microglia-like (MDMi) cells with pharmaceutical agents from the library of pharmaceutical agents to obtain indicators of the efficacy of the pharmaceutical agents; and b) using the indicators to select pharmaceutical agents from the library of pharmaceutical agents. Pharmaceutical agents may include a small molecule agent, a polypeptide agent, a protein agent, an antibody or antigen-binding fragment thereof agent, or a nucleic acid agent.

In certain aspects, provided herein are methods of identifying candidate neurodegenerative disease biomarkers, comprising the steps of: a) modifying and/or silencing a test gene in a population of human monocyte-derived microglia-like (MDMi) cells; b) obtaining an indicator of neuronal health in the MDMi cells; and c) using the indicator to identify the test gene as a candidate neurodegenerative disease biomarker.

In certain aspects, provided herein are methods of identifying candidate neurodegenerative disease biomarkers, comprising the steps of: a) modifying and/or silencing a test gene in a population of human monocyte-derived microglia-like (MDMi) cells; b) obtaining an indicator of neuronal health in the MDMi cells; and c) using the indicator to identify the test gene as a candidate neurodegenerative disease biomarker. In some embodiments, the test gene is silenced by contacting the population of MDMi cells using an inhibitory RNA molecule. In some embodiments, the test gene is modified or silenced by inducing a mutation in the genome of the cells of the population of MDMi cells. In some embodiments, the mutation in the genome of the cells is induced by contacting the population of MDMi cells with a CRISPR/Cas agent.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet, J. Chem. Inf. Model 45, 177-182, 2005). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: Chem Bridge, Princeton BioMolecular, Ambinter SARL, Enamine, ASDI, Life Chemicals etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about −2 to about 4) (see e.g., Angewante Chemie Int. ed. Engl. 24, 3943-3948, 1999). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see, e.g., Lipinski, J. Pharm. Tox. Methods 44, 235-249, 2000). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical successful if it is drug-like.

Several of these “drug-like” characteristics have been summarized into the four rules of Lipinski (generally known as the “rules of fives” because of the prevalence of the number 5 among them). While these rules generally relate to oral absorption and are used to predict bioavailability of compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to, cell lines or cultures described herein. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water or sterile saline each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see, e.g., Sambrook and Russel, Condensed Protocols from Molecular Cloning: A Laboratory Manual, Cold Spring Harbor Laboratory Press, 2006, ISBN-10: 0879697717; Ausubel et al., Short Protocols in Molecular Biology, 5th ed., Current Protocols, 2002 ISBN-10: 0471250929; Sambrook and Russel, Molecular Cloning: A Laboratory Manual, 3d ed., Cold Spring Harbor Laboratory Press, 2001, ISBN-10: 0879695773; Elhai and Wolk, Meth. Enzymol. 167:747-754, 1988; Studier, Protein Expr. Purif. 41:207-234, 2005; Gellissen, ed., Production of Recombinant Proteins: Novel Microbial and Eukaryotic Expression Systems, Wiley-VCH, 2005, ISBN-10: 3527310363; Baneyx, Protein Expression Technologies, Taylor & Francis, 2004, ISBN-10: 0954523253).

### Definitions

As used herein, “autologous” refers to a cell or cell type that is derived from the same individual.

As used herein, a “neurodegenerative disease” or “neurodegenerative disorder” refers to a disease or disorder that results in progressive loss of structure or function of neurons, including death of neurons. Such a disease may exhibit a number of symptoms, including, but not limited to, seizures, loss of motor and/or cognitive function, and the like. A neurodegenerative disease may encompass a CNS degenerative disease, or other diseases producing similar symptoms or having a common etiology. In some cases, orthologous genes in different species may exhibit similar mutations, thus resulting in similar diseases between species. For this reason, some species may serve as useful models for disease in other species.

The phrase “treating a neurodegenerative disease” refers to ameliorating the effects of, or delaying, halting or reversing the progress of, or delaying or preventing the onset of, a neurodegenerative disease as defined herein.

As used herein, a “therapeutic compound” refers to a molecule, such as a protein, peptide, polypeptide, a carbohydrate, an antibody or antibody fragment, a small molecule, or the like, that is not functionally present in cells of the subject or the like, and/or that will have a therapeutic benefit when delivered to cells of the subject.

As used herein, “subject” or “patient” refers to animals, including mammals, who are treated with the pharmaceutical compositions or in accordance with the methods described herein. As used herein, “pharmaceutically acceptable carrier,” “carrier,” “medium,” or “biologically compatible carrier” refers to reagents, cells, compounds, materials, compositions, and/or dosage forms that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio.

As used herein, “cell culture,” “in culture,” or “cultured” refers generally to cells taken from a living organism and grown under controlled conditions. A “primary cell culture” is a culture of cells, tissues, or organs taken directly from an organism before the first subculture. Cells are expanded in culture when they are placed in a growth medium under conditions that facilitate growth and/or division, resulting in a larger population of the cells. When cells are expanded in culture, the rate of cell proliferation is sometimes measured by the amount of time needed for the cells to double in number, referred to as “doubling time.”

As used herein, a “cell line” is a population of cells cultured in vitro formed by one or more sub-cultivations of a primary cell culture. Each round of sub-culturing is referred to as a passage. When cells are sub-cultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including, but not limited to, the seeding density, substrate, medium, growth conditions and time between passaging.

As used herein, “introducing,” “delivering,” and “administering” refer to the therapeutic introduction of a therapeutic compound to a subject. Administration may take place by any route.

As used herein, “regenerative” refers to the ability of a substance to restore, supplement, or otherwise rehabilitate the natural function of a tissue. This ability may be conferred by, for example, treating a dysfunctional tissue with a regenerative therapeutic compound. Regenerative compounds treat dysfunctional tissue by helping to restore the natural activity of dysfunctional tissue.

The terms “restore,” “restoration” and “correct” are used interchangeably herein and refer to the regrowth, augmentation, supplementation, and/or replacement of a defective tissue with a new and preferentially functional tissue. The terms include the complete and partial restoration of a defective tissue. Defective tissue is completely replaced if it is no longer present following the administration of the inventive composition. Partial restoration exists where defective tissue remains after the therapeutic composition is administered.

The phrase “effective amount” refers to a concentration or amount of a reagent, pharmaceutical composition, protein, cell population or other agent, that is effective for producing intended result, including treatment of neurodegenerative conditions, cell growth and/or differentiation in vitro or in vivo as described herein. An effective amount may vary depending on the specifics of the disorder to be treated, including but not limited to size or total volume/surface area to be treated, as well as proximity of the site of administration to the location of the region to be treated, among other factors familiar to one of skill.

As used herein, “functionally present” refers to a form or variant of a therapeutic compound as described herein, in which the compound is not functional in the subject or cell. For example, a cell or subject may comprise a specific gene, gene sequence, protein, polypeptide, or the like, but that gene, gene sequence, protein, polypeptide, or the like is non-functional, absent, or otherwise inactive. The methods and compositions of the present disclosure may provide a supplemental or replacement form or variant of such a compound in order to restore or provide the intended function of the compound.

A “clone,” or “clonal cell,” is a line of cells that is genetically identical to the originating cell. This cloned line is produced by cell division (mitosis) of the originating cell. The term “clonal population” in reference to the cells of the invention shall mean a population of cells that is derived from a clone. A cell line may be derived from a clone and is an example of a clonal population.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1

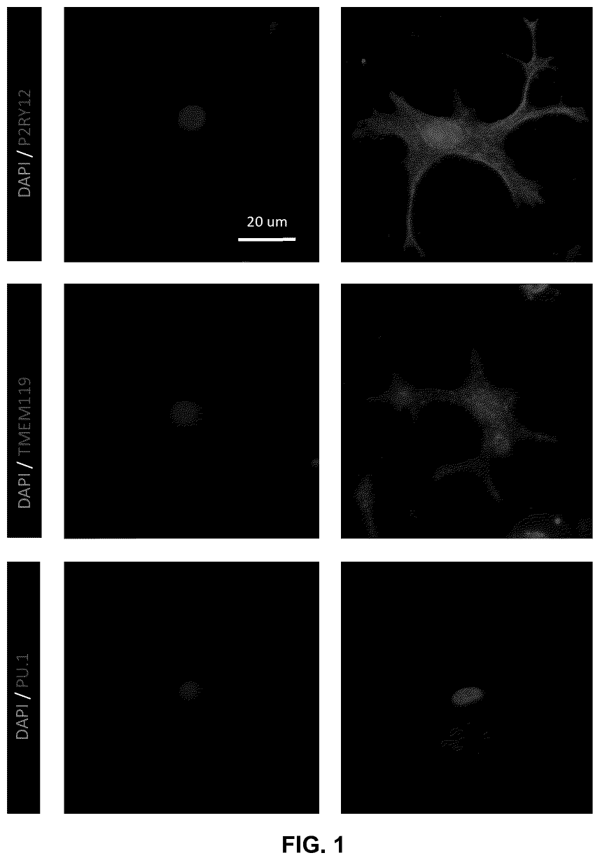

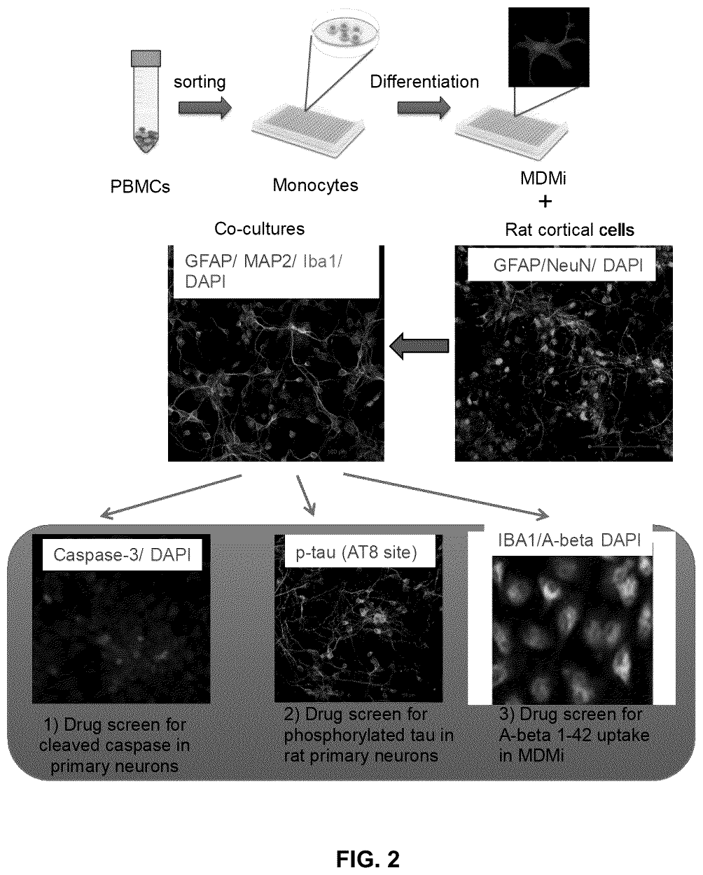

This example shows recapitulation of the in vivo physio-pathological pathways of microglia using an in vitro model of human monocyte-derived microglia-like cells (MDMi) (FIG. 1) co-cultured with rat primary neuronal cells in a 96-well plate. An efficient in vitro model for the screen of small molecules and biologics targeting human microglia and their influence on neuronal function is provided. This co-culture can be extended for 14-20 days and microglia uptake function, neuronal viability and synaptic pruning in response to stress (e.g., amyloid peptides, alpha-synuclein, etc.) are measured using a four-color imaging system (FIG. 2). To study the influence of inflammatory milieu from patients on microglia and neuronal function, patients' sera can be added to the co-culture for 24-48 hours.

### Generation of Monocyte-Derived Microglia-Like Cells (MDMi) (FIG. 1):

Generation of MDMi cells were prepared as described in Ryan K J, White C C, Patel K, et al. A human microglia-like cellular model for assessing the effects of neurodegenerative disease gene variants. Sci Transl Med. 2017;9(421):eaai7635. Frozen or fresh peripheral blood mononuclear cells (PBMCs) can be used as a source of monocytes. PBMCs are separated by Lymphoprep gradient centrifugation (StemCell Technologies) from healthy or diseased subjects. PBMCs are frozen at a concentration of 1-3×10⁷ cells ml⁻¹ in 10% DMSO (Sigma-Aldrich)/90% fetal bovine serum (vol/vol, Corning). Prior to each study, aliquots of frozen PBMCs are thawed and washed in 10 ml PBS. Monocytes are positively selected from whole PBMCs using anti-CD14+ microbeads (Miltenyi Biotech) and plated at the following densities per well: 1×10⁵ cells (96-well plate), 3×10⁵ cells (24-well plate), 1×10⁶ cells (6-well plate) in temperature-sensitive Nunc plates with Upcell surface (ThermoFisher). To induce the differentiation of MDMi, monocytes are incubated in serum-free conditions using RPMI-1640 Glutamax (Life Technologies) with 1% penicillin/streptomycin (Lonza) and 2.5 μg/ml Fungizone (Life Technologies) and a mixture of the following human recombinant cytokines: M-CSF (10 ng/ml; Biolegend 574806), GM-CSF (10 ng/ml; R&D Systems 215-GM-010/CF), NGF-β (10 ng/ml; R&D Systems 256-GF-100), CCL2 (100 ng/ml; Biolegend 571404) and IL-34 (100 ng/ml; R&D Systems 5265-IL-010/CF) at standard humidified culture conditions (37° C., 5% CO₂) for 10-15 days.

Rat/Mouse Primary Neuronal Cultures: Primary neuronal cultures are made from cerebral cortices of Sprague Dawley or Wistar rat embryos (day 17 of gestation) or mouse embryos (day 16 of gestation). Cortices from each embryo are dissected and mechanically dissociated with a Pasteur pipette. The cell suspension is then centrifuged at 250 g for 10 min and resuspended in culture medium. To express reporter genes that can be used as readouts of senescence and neurodegeneration (i.e., genes associated with neuronal apoptosis, synaptic pruning, etc.), dissociated cells are transfected by electroporation using Amaxa or Neon system with the desired marker tagged with a fluorescent marker. Cells will be cultured in Corning 96-well black polystyrene plates (Sigma-Aldrich) at a density of 1×10⁵ cells precoated with poly-L-lysine (5 μg/ml) and laminin (4 μg/ml). Culture medium consisted of Neurobasal medium (Gibco, Grand Island, NY) complemented with B-27 supplement, 0.5 mM glutamine, inactivated fetal calf serum (1%), and inactivated horse serum (1%).

iPSC-derived neurons: The protocol used to differentiate iPSCs to cortical neurons follows that published previously (PMID: 24465796). iPSCs are cultured in feeder-free conditions using mTeSR1 media (Stem Cell Technologies) and plates coated with matrigel (Corning). Media are replenished every two days. iPSCs are passaged every 5-7 days using ReLeSR (Stem Cell Technologies, Cambridge, MA). Cortical Neural progenitor cells (NPCs) are derived from the cell line. Cryopreservation, maintenance, characterization and expansion of cortical NPCs are carried out according to previously published protocol (PMID: 24465796). Human cortical neurons are differentiated from cortical neural precursor cells as described earlier (PMID: 24465796).

Generation of Pro- and anti-Inflammatory Human T cells: Venous blood is drawn from healthy control volunteers. Ficoll-Paque PLUS (GE Healthcare) is used to separate PBMCs by gradient centrifugation. Naive CD4⁺ T cells are subsequently isolated from fresh PBMCs by negative selection with beads using the CD4⁺ T-cell isolation kit II (Miltenyi). Cells are cultured at 10⁴ cells per well in serum-free X-Vivo 15 media (Lanza), and stimulated far 7 days with plate-bound anti-CD3 (UCHT1, 5 μg ml⁻¹) and soluble anti-CD28 (28.2, 1 μg ml⁻¹) Abs in the presence of anti-IFN-γ plus anti-IL-4 for neutral T helper cells (Th0), 1L-12 (10 ng ml⁻¹) plus anti-IL-4 for T helper 1 (Th1), TGF-β1 (3 ng ml⁻¹), IL-6, IL-21, IL-23 (all at 25 ng ml⁻¹) and IL-β1 (12.5 ng ml⁻¹) for T helper 17 cells (Th17), or TGF-β1 (5 ng ml⁻¹) for inducible regulatory T cells (iTregs). Neutralizing Abs were used at 10 μg ml⁻¹. All recombinant proteins are purchased from R&D Systems. Neutralizing antibodies are purchased from BD Biosciences.

Preparation of Serum and Plasma from Patients and Controls: Blood samples are collected into EDTA-treated tubes for plasma or plain tubes for serum. Samples are centrifuged for at least 15 minutes at 2200-2500 RPM. Serum or plasma are aliquoted into a clean plastic vial and stored at −80° C.

Neuronal-MDMi (FIG. 2): To culture human microglia-like cells with rat primary neuronal cultures, these two cell types are generated separately as described above, then MDMi culture are de-attached from the temperature-sensitive plates and then added to the neuronal cultures on day 10-14 after culture at 1:10 ratio (Neuron:MDMi).

### Applications

Among other applications, the MDMi-neuron co-culture is a useful model to screen the effect of medicinal small molecules and biologics (monoclonal antibodies, ligands, fusion proteins) on microglia phagocytic function and the consequence of microglia manipulation on neuronal health and synaptic density. Additionally, to add the peripheral immunity arm to the co-culture, patients' sera or peripheral immune cells can be added to the co-culture to measure the influence of peripheral immunity on the CNS.

### Screen of Small Molecule Screen and Biologics

The presently disclosed culture model consists of human microglia-like cells polarized from human monocytes isolated from genotyped subjects, and co-cultured with either rodent primary neurons or with iPSC-derived neurons in the presence of human fluids such as serum, plasma or CSF collected from Alzheimer's disease patients (or patients having other neurodegenerative diseases) or healthy controls. The cultures are grown in a 96-well plate for up to 3 weeks followed by the analysis of microglia phagocytic activity, neuronal synaptic density and cell viability using a 4-color imaging system (Celigo™) that is able to scan a 96-well plate in seven minutes. For additional information on gene targeted by the screen, RNA is collected and subjected to RNA sequencing analysis. For co-culturing MDMi with human iPSC-derived neurons, MDMi and human neurons are first differentiated in separate plates where MDMi are differentiated on temperature-responsive UpCell plates (Thermo Scientific™ Nunc™ UpCell™ Dishes) then transferred to human neuronal cultures by the end of their differentiation.

To test the effects of patients' serum/plasma, CSF or lymphocytes the following method is performed:

i: Serum/plasma/CSF: Serum, plasma or CSF from AD, ALS, PD or any other neurodegenerative disease are added to the culture media of MDMi-neuron (25% of total volume or 50 μl of serum or plasma+150 μl of culture media) for 48 hrs, followed by analysis if MDMi phagocytic activity. Neuronal integrity (tau phosphorylation, viability, and synaptic formation) is also assessed.

ii: CD3+ T cells: T cell lymphocytes are isolated from PBCMs of patients by flow sorting and are added to neuron-MDMi cultures at the following ratio: 10:1:1, followed by assessment of MDMi uptake function and neuronal integrity after 48 hours.

### shRNA Knockdown Screen

To study the effect of microglia related genes on neuronal health, the model described above is utilized. In brief, gene knockdown is performed in MDMi using shRNA approach, and gene knockdown is confirmed by quantitative PCR. Successful MDMi cultures are then co-incubated with primary cortical neurons in the presence or absence of disease-related stress (i.e., aggregated amyloid peptides in the case of an AD study), and neuronal cultures are evaluated.

### Example 2

In vitro validation of repurposed drugs in microglia, astrocyte and neuron co-cultures.

In order to determine the efficacy, validity and therapeutic potential in AD pathology, neuronal apoptosis (cleaved caspase-3), amyloid-beta uptake and the ratio of phosphorylated-tau/total tau are measured in a 3-way cellular model after treatment with potential therapeutic agents. In this three cell-type model, a system for examining AD relevant functions has been optimized. This system is used to carefully investigate the efficacy and toxicity potential AD therapeutic agents.

The therapeutic agents are screened in triplicate. 50,000 human monocytes are plated in 96-well plates and polarized to microglia-like cells using M-CSF, GM-CSF, NGF-β, CCL2 and IL-34 for 10 days in serum free medium. At day 10, 100,000 embryonic day-17 cortical cells are added to the MDMi cultures. Generation of MDMi cells were prepared as described in Ryan K J, White C C, Patel K, et al. A human microglia-like cellular model for assessing the effects of neurodegenerative disease gene variants. Sci Transl Med. 2017;9(421):eaai7635. Seven days later the cultures are stressed with aggregated recombinant amyloid-beta (10 μg/ml), and treated with the therapeutic agents at a final concentration of 1 μM for 24 hours.

Cultures are imaged using the Celigo imaging system to measure: i) Uptake of amyloid-beta by co-staining for IBA1 and anti-Amyloid-beta. ii) Tau phosphorylation: Cells are stained using ATF8 antibody that detects phosphorylated tau on the serine 202/Threonine 205 epitope. iii) Apoptosis: Apoptotic cell death is measured in the co-cultures by co-staining each cell marker [anti-,MAP2 antibody (neuronal marker), GFAP (astrocyte marker), IBA1 (microglia marker)] with anti-cleaved caspase-3 antibody.

In this study, therapeutic agents are identified that may be 1) affecting one cell type, 2) affecting multiple cell types independently or 3) affecting how the cell types communicate. To this end, a robust co-culture system has been developed where rat primary cortical cultures prepared from embryonic day 17 that includes ˜80% neurons, 10-20% astrocytes on Day 10 MDMi are grown for 7 days to create a multi-cellular system.

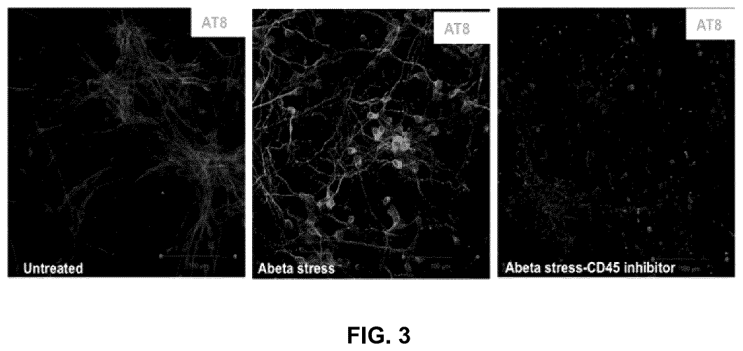

It was shown that adding a microglia specific small molecule that inhibits CD45 (only expressed on immune cells) can decrease amyloid-beta stress induced phosphorylation of tau protein (FIG. 3).

### Example 3

1. Comparison of the effects of serum from medically-refractory and medically-controlled epilepsy patients on the phenotype and function of healthy MDMi.

In order to study the effects of patient peripheral inflammatory milieu on microglia phenotype and function, the effects of serum from medically-refractory and medically-controlled epilepsy patients on healthy MDMi cultures was compared by two assays: Cytokine profile of MDMi cells and MDMi cell uptake function.

a. Effects on cytokine and chemokine production by MDMi:

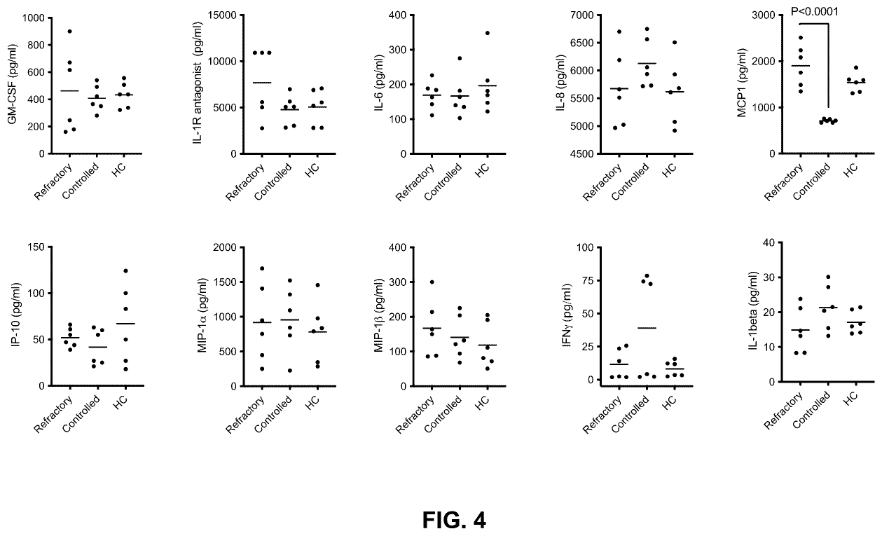

MDMi cells were prepared as described in Ryan K J, White C C, Patel K, et al. A human microglia-like cellular model for assessing the effects of neurodegenerative disease gene variants. Sci Transl Med. 2017;9(421):eaai7635. Healthy MDMi cells were exposed to patient sera as well as sera from healthy controls at 1:4 ratio. Following 24 hours of treatment, media were collected, and a panel of cytokine and chemokines (GM-CSF, IL-1R, IL-6, IL-8, MCP1, IP-10, MIP-1alpha, MIP-1beta, IFNgamma, IL-1beta) were measured by Luminex assay. FIG. 4 shows a significant increase in MCP1 in medically-refractory patients following treatment.

b. Effects on uptake function of MDMi cells.

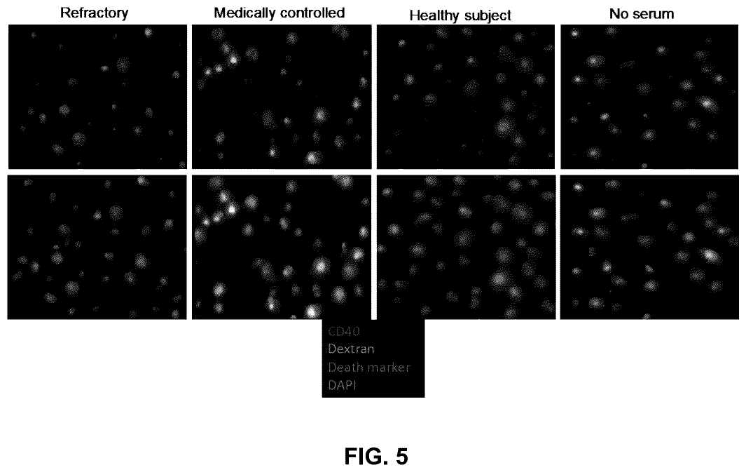

Generation of MDMi cells were prepared as described in Ryan K J, White C C, Patel K, et al. A human microglia-like cellular model for assessing the effects of neurodegenerative disease gene variants. Sci Transl Med. 2017;9(421):eaai7635. Healthy MDMi were exposed to patient sera as well as from healthy controls at 1:4 ratio. Following 24 hours of treatment, MDMi were exposed to FITC-labelled Dextran for 2 hours, MDMi cultures were rinsed, stained with an innate immune cell marker, CD40, in addition to the dead cell dye (AO/PI) and a nuclear stain (DAPI). Plates were imaged using high throughput imaging reader followed by cell lysing with RNA lysis buffer and cells were kept in the −80° C. freezer for future RNA sequencing analysis. For the analysis of MDMi uptake function, random regions of MDMi cultures were selected and the number CD40+ cells overlapping with dextran staining were analyzed using FIJI software. FIG. 5 shows uptake in medically-controlled patients after treatment.

2. Comparing the effects of CSF from Alzheimer's disease subjects on uptake function.

In order to perform population stratification in Alzheimer's disease, MDMi cells were generated from healthy subjects and were exposed to patient cerebrospinal fluid (CSF). Generation of MDMi cells were prepared as described in Ryan K J, White C C, Patel K, et al. A human microglia-like cellular model for assessing the effects of neurodegenerative disease gene variants. Sci Transl Med. 2017;9(421):eaai7635. Briefly CSF was added to MDMi culture media at 1:4 ratio for 24 hours, followed by the addition of AF488-labeld amyloid 1-42 peptide for 2 hours. Cells were then stained using cellTracker CMPTX dye and a nuclear dye, DAPI. Cells were rinsed and imaged and analyzed as described above. Cytochalasin D were used a negative control of uptake function.

### INCORPORATION BY REFERENCE

All publications patent applications mentioned herein are hereby incorporated by reference in their entirety as if each individual publication or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of assessing the effect of a clinical intervention on a human subject, the method comprising the steps of
a) obtaining a population of human monocyte-derived microglia-like (MDMi) cells from a human subject undergoing a clinical intervention;
b) profiling the population of MDMi cells to obtain an indicator of the effect of the clinical intervention; and
c) using the indicator to assess the effect of the clinical intervention.

2. The method of claim 1, further comprising the step of co-culturing the population of MDMi cells from the human subject with a population of different cells.

3. The method of claim 2, wherein the population of different cells comprise neuron cells, astrocyte cells, T cells, oligodendrocyte cells, endothelial cells, or tumor cells.

4. The method of claim 2, wherein step b) further comprises profiling the population of different cells to obtain the indicator.

5. The method of claim 1, wherein the subject has a neurodegenerative disorder.

6. The method of claim 5, wherein the neurodegenerative disorder is Alzheimer's disease (AD), amyotrophic lateral sclerosis (ALS), Parkinson's disease (PD), Huntington's disease (HD), epilepsy, frontotemporal dementia (FTD), Friedreich ataxia, Lewy body disease, Spinal muscular atrophy, Multiple Sclerosis, Progressive Multifocal Leukoencephalopathy, or Leukodystrophy.

7. The method of claim 1, wherein step a) comprises deriving the MDMi cells from monocytes obtained from the subject.

8. The method of claim 1, wherein the population MDMi cells are further differentiated into cells resembling microglial subtypes prior to step b).

9. The method of claim 1, wherein the clinical intervention at least one of
is selected from cell-based therapy, small molecule therapy, biologic therapy, or surgery, or comprises the administration of a pharmaceutical agent to the subject.

10. (canceled)

11. The method of claim 10, wherein the pharmaceutical agent is selected from a small molecule agent, a polypeptide agent, a protein agent, an antibody or antigen-binding fragment thereof agent, and a nucleic acid agent.

12. The method of claim 1, wherein the indicator comprises measures of protein expression, transcription, epigenetic modifications, uptake ability, cytokine production, chemokine production, cell morphological analysis, cell trafficking, or cell viability.

13. The method of claim 1, wherein step c) comprises comparing the indicator to a reference indicator.

14. The method of claim 13, wherein the reference indicator at least one of
is an indicator obtained from the subject at an earlier time, or
was obtained from the subject prior to the start of the clinical intervention.

15. (canceled)

16. The method of claim 1, wherein if the clinical intervention is determined to be effective in step c) then the clinical intervention continues to be administered to the subject.

17. The method of claim 1, wherein if the clinical intervention is determined to be ineffective in step c) then at least one of
the clinical intervention is no longer administered to the subject, or
a different clinical intervention is administered to the subject.

18. (canceled)

19. The method of claim 1, wherein the first population monocyte-derived microglia-like (MDMi) cells from the human subject undergoing or about to undergo the clinical intervention is a first population and the indicator is a first indicator,
and further comprising subjecting the subject to the clinical intervention;
obtaining a second population MDMi cells from the human subject undergoing the clinical intervention;
profiling the second population of MDMi cells to obtain a second indicator; and
wherein using the first indicator comprises comparing the first indicator with the second indicator to assess the effect of the clinical intervention.

20. The method of claim 19, further comprising the step of co-culturing the first population of MDMi cells from the human subject and/or the second population of MDMi cells from the human subject with a population of different cells.

21-25. (canceled)

26. The method of claim 19, wherein the first population of MDMi cells are further differentiated into cells resembling microglial subtypes prior to profiling the first population and/or the second population of MDMi cells are further differentiated into cells resembling microglial subtypes prior to profiling the second population.

27-51. (canceled)

52. A method of determining whether a human subject is likely to respond to a pharmaceutical agent, the method comprising the steps of:
a) obtaining a population of human monocyte-derived microglia-like (MDMi) cells from a human subject; and
b) contacting the MDMi cells with a pharmaceutical agent to obtain an indicator of whether the subject is likely to respond to the pharmaceutical agent; and
c) using the indicator to determine whether the subject is likely to respond to the pharmaceutical agent.

53-64. (canceled)

65. The method of claim 1, wherein obtaining a population of human monocyte-derived microglia-like (MDMi) cells comprises
culturing the population of human monocyte-derived microglia-like (MDMi) cells with a biomaterial from the human subject undergoing the clinical intervention.

66-82. (canceled)

83. The method of claim 1, wherein the population monocyte-derived microglia-like (MDMi) cells from the human subject undergoing or about to undergo a clinical intervention is a first population and indicator is a first indicator, and wherein obtaining the first population monocyte-derived microglia-like (MDMi) cells comprises
culturing the first population monocyte-derived microglia-like (MDMi) cells with a first biomaterial from the human subject undergoing or about to undergo the clinical intervention;
and further comprising culturing a second population MDMi cells with a second biomaterial from the human subject undergoing the clinical intervention;
profiling the second population of MDMi cells to obtain a second indicator; and
wherein using the first indicator comprises comparing the first indicator with the second indicator to assess the effect of the clinical intervention.

84-116. (canceled)

117. The method of claim 52, wherein obtaining a population of human monocyte-derived microglia-like (MDMi) cells comprises
culturing the population of human monocyte-derived microglia-like (MDMi) cells with a biomaterial from the human subject.

118-165. (canceled)

166. A method of identifying a therapeutic agent for treatment of a neurodegenerative disease, the method comprising:
a) co-culturing a human monocyte-derived microglia-like cell with rodent or human-derived primary neuronal cells in the presence of a fluid sample from a patient with a neurodegenerative disease;
b) contacting test compounds with the product of step a); and
c) identifying a test compound that protects or increases synaptic formation, regulates synaptic pruning or microglia uptake function or neuronal viability in comparison to a control compound.

167-174. (canceled)
